(19) United States
(12) Patent Application Publication
(10) Pub. No.: US 2024/0366189 A1
(43) Pub. Date: Nov. 7, 2024

(54) ULTRASOUND DIAGNOSTIC APPARATUS AND TRANSMISSION BEAM FORMING METHOD

(71) Applicant: FUJIFILM Healthcare Corporation, Kashiwa-shi (JP)

(72) Inventor: Masanori Hisatsu, Chiba (JP)

(21) Appl. No.: 18/639,544

(22) Filed: Apr. 18, 2024

(30) Foreign Application Priority Data
May 1, 2023 (JP) 2023-075809

(51) Int. Cl. A61B 8/00; G01S 7/52; G01S 15/89

(52) U.S. Cl. CPC A61B 8/54; A61B 8/4488; G01S 7/5202; G01S 15/8915

(57) ABSTRACT

A weakness of a first transmission weighting method is complemented with a second transmission weighting method. A transmission aperture includes an intermediate portion, a first end portion, and a second end portion. A main transmission weighting method of varying a transmission voltage is applied to a plurality of transmission signals supplied to the intermediate portion. Voltages of a plurality of transmission signals supplied to the first end portion and the second end portion are all a constant value, and a sub-transmission weighting method of varying a transmission pulse width or a wave number is applied to the transmission signals.

### BACKGROUND OF THE INVENTION

### 1. Field of the Invention

The present disclosure relates to an ultrasound diagnostic apparatus and a transmission beam forming method, and particularly to generation of a transmission signal string.

### 2. Description of the Related Art

An ultrasound diagnostic apparatus is used in an ultrasound examination of a subject. The ultrasound diagnostic apparatus includes an ultrasound probe comprising an oscillator array. In a case where the ultrasound waves are transmitted, a transmission aperture is set for the oscillator array, and a transmission signal string is supplied to an oscillator string constituting the transmission aperture. Accordingly, a transmission beam is radiated from the transmission aperture to an inside of a living body.

In order to form the transmission beam having a favorable form, specifically, for example, in order to reduce unnecessary sidelobes and to make a transmission beam width uniform in a depth direction, transmission apodization is performed. That is, the power of the ultrasound waves radiated from each oscillator in the transmission aperture is adjusted such that a desired sound pressure distribution is generated in the transmission aperture. In the present specification, the transmission apodization will be referred to as transmission weighting or simply weighting.

As a general transmission weighting method, a weighting method of varying a transmission voltage is known. In the method, the voltage of each transmission signal supplied to the transmission aperture is determined in accordance with a weight curve.

U.S. Pat. No. 6,135,963A and US2020/0393420A disclose a method of performing transmission weighting by pulse width modulation (PWM). JP2005-319177A discloses an ultrasound diagnostic apparatus that performs contrast harmonic imaging (CHI). In the CHI, a voltage of an entire transmission signal string is lowered in order not to burst bubbles as a contrast medium or in order not to burst the bubbles more than necessary.

### SUMMARY OF THE INVENTION

In a case where only one transmission weighting method is used for the transmission weighting, an ideal sound pressure distribution may not be formed. For example, in a case of execution of the transmission weighting method of varying the transmission voltage, a low transmission voltage may not be correctly generated due to a restriction derived from a transmission circuit or the like. In such a case, an ideal sound pressure distribution cannot be formed.

An object of the present disclosure is to generate an ideal or appropriate sound pressure distribution in a living body in a case of formation of a transmission beam. Alternatively, an object of the present disclosure is to complement a weakness of a first transmission weighting method with a second transmission weighting method.

The present disclosure relates to an ultrasound diagnostic apparatus including: an oscillator array; a transmission circuit that supplies a transmission signal string to a transmission aperture set in the oscillator array; and a controller that controls generation of each transmission signal constituting the transmission signal string, in which the transmission aperture has a first portion and a second portion, and the controller applies a first transmission weighting method to a plurality of transmission signals supplied to the first portion, and applies a second transmission weighting method different from the first transmission weighting method to a plurality of transmission signals supplied to the second portion.

The present disclosure relates to a transmission beam forming method including: a generation step of generating a transmission signal string for forming a transmission beam; and a supply step of supplying the transmission signal string to a transmission aperture set in an oscillator array, in which the transmission aperture has a first portion and a second portion, and in the generation step, a first transmission weighting method is applied to a plurality of transmission signals supplied to the first portion, and a second transmission weighting method different from the first transmission weighting method is applied to a plurality of transmission signals supplied to the second portion.

According to the present disclosure, an ideal or appropriate sound pressure distribution is generated in the living body in a case of formation of the transmission beam. Alternatively, according to the present disclosure, a weakness of the first transmission weighting method can be complemented with the second transmission weighting method.

### BRIEF DESCRIPTION OF THE DRAWINGS

FIG. 1 is a block diagram showing an ultrasound diagnostic apparatus according to an embodiment.

FIG. 2 is a diagram showing a transmission weighting method according to the embodiment.

FIG. 3 is a diagram showing a plurality of frequency characteristics.

FIG. 4 is a diagram showing a plurality of sound pressure waveforms.

FIG. 5 is a diagram showing a first example of three transmission signals.

FIG. 6 is a diagram showing a second example of the three transmission signals.

FIG. 7 is a flowchart showing transmission/reception control according to the embodiment.

### DESCRIPTION OF THE PREFERRED EMBODIMENTS

Hereinafter, an embodiment will be described with reference to the accompanying drawings.

### (1) Outline of Embodiment

An ultrasound diagnostic apparatus according to the embodiment includes an oscillator array, a transmission circuit, and a controller. The transmission circuit supplies a transmission signal string to a transmission aperture set in the oscillator array. The controller controls generation of each transmission signal constituting the transmission signal string. The transmission aperture includes a first portion and a second portion. The controller applies a first transmission weighting method to a plurality of transmission signals supplied to the first portion, and applies a second transmission weighting method different from the first transmission weighting method to a plurality of transmission signals supplied to the second portion.

With the above-described configuration, even in a case where it is difficult to apply the first transmission weighting method to the second portion, the second transmission weighting method can be applied to the second portion. Therefore, a sound pressure distribution formed by the transmission aperture can be made appropriate. Accordingly, for example, unnecessary sidelobes can be reduced, and a transmission beam width can be made uniform in a depth direction.

In the embodiment, the first portion is an intermediate portion in the transmission aperture, and the second portion is a first end portion and a second end portion provided on both sides of the intermediate portion in the transmission aperture. In the embodiment, the first transmission weighting method is a weighting method of varying a transmission voltage (first transmission parameter), and the second transmission weighting method is a weighting method of varying a transmission parameter (second transmission parameter) other than the transmission voltage.

In a case where the transmission weighting method of varying the transmission voltage is used, it is required to reduce the voltages of a plurality of transmission signals supplied to both end portions of the transmission aperture. In this case, it may be required to generate a voltage lower than a voltage range that can be stably generated. In a case where the second transmission weighting method of not depending on the variation of the transmission voltages to the plurality of transmission signals supplied to both end portions, that is, varying the transmission parameter other than the transmission voltage is applied, an ideal sound pressure distribution can be realized over the entire transmission aperture.

In the embodiment, in the first transmission weighting method, the transmission voltage is varied over a range from a first transmission voltage to a second transmission voltage lower than the first transmission voltage. The voltages of the plurality of transmission signals supplied to the first end portion and the second end portion are all the second transmission voltage. Waveforms of the plurality of transmission signals supplied to the first end portion and the second end portion are set in accordance with the second transmission weighting method.

The first transmission voltage is, for example, a transmission voltage (maximum transmission voltage) designated by a user or automatically determined. The second transmission voltage is, for example, a minimum transmission voltage determined in advance in consideration of the characteristics of the transmission circuit and the like. After the voltages of the plurality of transmission signals supplied to the first end portion and the second end portion are fixed to the second transmission voltage, the transmission weighting of varying the parameter other than the transmission voltage is applied to the plurality of transmission signals.

It should be noted that, although the voltages of the plurality of transmission signals supplied to the first end portion and the second end portion are fixed to the second transmission voltage, the second transmission voltage is set in accordance with the first transmission weighting method, and thus it is also possible to understand that both the first transmission weighting method and the second transmission weighting method are applied to the plurality of transmission signals supplied to the first end portion and the second end portion.

In the embodiment, the transmission aperture includes a first boundary between the intermediate portion and the first end portion, and a second boundary between the intermediate portion and the second end portion. The controller controls variation of the transmission voltage and variation of the transmission waveform such that a convex sound pressure distribution (sound pressure curve) corresponding to the transmission aperture is substantially continuous at the first boundary and the second boundary.

In the embodiment, the second transmission parameter is a pulse width. The controller varies the pulse width of each transmission signal in a case of application of the second weighting method. Alternatively, the second transmission parameter is a wave number. The controller varies the wave number of the transmission signal in a case of application of the second transmission weighting method. By varying the pulse width and varying the wave number, the power of the ultrasound pulse radiated from the oscillator array is changed. The wave number is the number of waves constituting one transmission signal.

In the embodiment, the controller applies the first transmission weighting method to the plurality of transmission signals supplied to the entire transmission aperture in a case where a representative transmission voltage of the transmission signal string is higher than a threshold value. In a case where the representative transmission voltage is lower than the threshold value, the first transmission weighting method is applied to the plurality of transmission signals supplied to the intermediate portion, and the second transmission weighting method is applied to the plurality of transmission signals supplied to the first end portion and the second end portion.

With the above-described configuration, in a case where an appropriate sound pressure distribution can be formed by using only the first transmission weighting method, the transmission control is simplified. On the other hand, in a case where an appropriate sound pressure distribution cannot be formed only by the first transmission weighting method, an appropriate sound pressure distribution can be formed by using the second transmission weighting method in combination. The representative transmission voltage is, for example, the transmission voltage (maximum transmission voltage) designated by the user or automatically determined. Another voltage may be determined as the representative transmission voltage. Alternatively, it may be determined whether to use only the first transmission weighting method or to use both the first transmission weighting method and the second transmission weighting method, in accordance with a transmission mode or a transmission condition.

A transmission beam forming method according to the embodiment includes a generation step of generating a transmission signal string for forming a transmission beam, and a supply step of supplying the transmission signal string to a transmission aperture set in an oscillator array. The transmission aperture includes a first portion and a second portion. In the generation step, a first transmission weighting method is applied to a plurality of transmission signals supplied to the first portion, and a second transmission weighting method different from the first transmission weighting method is applied to a plurality of transmission signals supplied to the second portion.

In a case where the first transmission weighting method and the second transmission weighting method are applied to the entire transmission aperture, the transmission control is quite complicated. In contrast, in a case where the first transmission weighting method and the second transmission weighting method are limitedly applied to a part of the transmission aperture, the transmission control is simplified. Since the transmission voltage can be generally easily varied, the voltage varying method is adopted as the first transmission weighting method in the embodiment. A method other than the voltage varying method is adopted as the second transmission weighting method.

### (2) Details of Embodiment

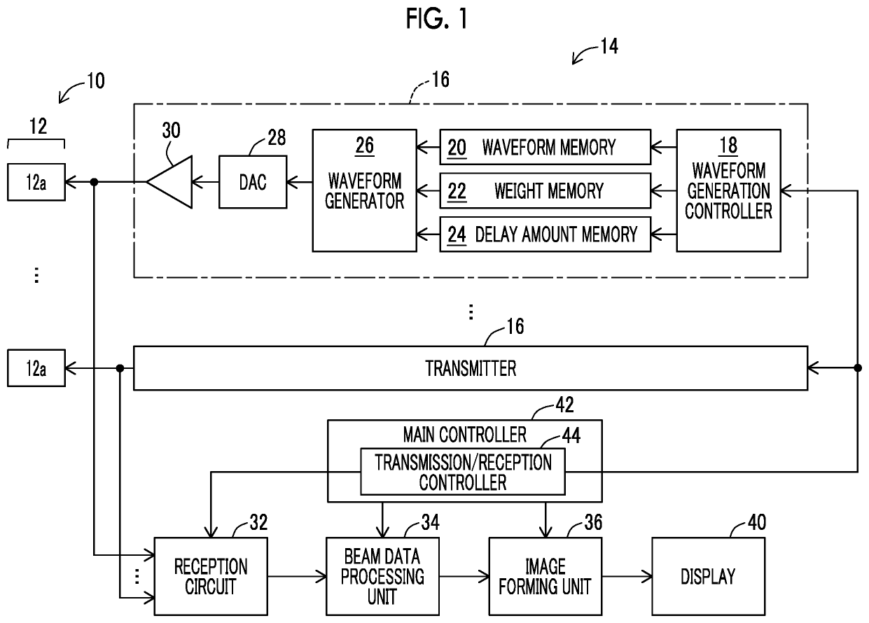

FIG. 1 shows a configuration example of the ultrasound diagnostic apparatus according to the embodiment. This ultrasound diagnostic apparatus is used for an ultrasound examination of a subject. The ultrasound diagnostic apparatus has several transmission/reception modes (operation modes). Among these, a contrast harmonic imaging (CHI) mode that images a contrast medium injected into the subject is included.

An ultrasound probe 10 includes an oscillator array 12. The oscillator array 12 is configured with a plurality of oscillators 12a. An ultrasound beam is formed by the oscillator array 12, and electronic scanning with the ultrasound beam is performed. As an electronic scanning method, an electronic linear scanning method, an electronic sector scanning method, or the like is known. In the electronic linear scanning method, apertures (transmission aperture and reception aperture) are set for the oscillator array 12, and the aperture is electronically scanned. In the electronic sector scanning method, the apertures (transmission aperture and reception aperture) are set for the entire oscillator array 12.

A transmission circuit 14 is an electronic circuit that functions as a transmission beam former. The transmission circuit 14 is configured with a plurality of transmitters 16 that output the plurality of transmission signals in parallel.

The transmitters 16 have the same configuration. In the embodiment, the transmitter 16 includes a waveform generation controller 18, a waveform memory 20, a weight memory 22, a delay amount memory 24, a waveform generator 26, a DAC 28, and a linear amplifier 30. The transmission waveform is stored in the waveform memory 20. In the transmission weighting, in a case where a plurality of transmission waveforms are used, the transmission waveforms are stored in the waveform memory 20 in advance. A weight is stored in the weight memory 22. Specifically, a voltage value is stored as the weight. The weight memory 22 may store the pulse width (duty) or the wave number, which will be described later, as the weight. The delay amount memory 24 stores a delay amount. The waveform generation controller 18 controls writing of each data in each of the memories 20, 22, and 24.

The waveform generator 26 generates the transmission signal by applying the weight read from the weight memory 22 and applying the delay amount read from the delay amount memory 24, to the waveform data read from the waveform memory 20. The application of the weight is usually a setting of the transmission voltage. In a case where the transmission signal is weighted by varying the duty or the wave number, the waveform data corresponding to the designated weight is read from the waveform memory 20.

The DAC 28 is a converter that converts a digital signal into an analog signal. The transmission signal as the digital signal is input to the DAC 28. The transmission signal as the analog signal is output from the DAC 28. The linear amplifier 30 amplifies the transmission signal. The amplified transmission signal is supplied to the oscillator 12a corresponding to the transmission signal.

The plurality of transmitters 16 have a function of applying the weighting to the plurality of transmission signals supplied to the transmission aperture. In each transmitter 16, the portions related to the weighting are the waveform generation controller 18, the waveform memory 20, the weight memory 22, and the waveform generator 26. A transmission/reception controller 44, which will be described later, is also involved in the weighting. Among these configurations, the waveform generation controller 18 and the transmission/reception controller 44 correspond to a controller or a control unit in terms of the transmission weighting.

During the reception, a plurality of reception signals output in parallel from the reception aperture are transmitted to a reception circuit 32. The reception circuit 32 is an electronic circuit that functions as a reception beam former, and includes a plurality of receivers. Each receiver includes a preamplifier, an ADC, a delay unit, and the like. The reception circuit 32 includes an adder that adds the plurality of reception signals output from a plurality of delay units. That is, in the reception circuit 32, a phase alignment addition is applied to the plurality of reception signals, to generate reception beam data.

A beam data string is generated in accordance with the electronic scanning with the ultrasound beam, and the beam data string is input to a beam data processing unit 34. The beam data processing unit 34 includes a detector, a logarithmic converter, and the like. An image forming unit 36 forms an ultrasound image based on the beam data string output from the beam data processing unit 34. The ultrasound image is, for example, a B-mode tomographic image. The image forming unit 36 includes, for example, a digital scan converter (DSC). The ultrasound image is displayed on a display 40.

A main controller 42 is configured with a CPU that executes a program. The main controller 42 controls the operations of the components shown in FIG. 1. The main controller 42 functions as the transmission/reception controller 44. The transmission/reception controller 44 controls the operations of the plurality of transmitters 16. Specifically, the operations of the plurality of waveform generation controllers 18 provided in the plurality of transmitters 16 are controlled, and the transmission weighting is applied to the plurality of transmission signals through this control. In addition, the transmission/reception controller 44 according to the embodiment has a function of generating the plurality of transmission waveforms, a function of calculating a weight distribution, and a function of calculating a delay value distribution.

In a harmonic imaging mode including the CHI mode, for example, in accordance with a pulse inversion (PI) method, a normal phase transmission signal string and a reversed phase transmission signal string are supplied to the transmission aperture in order. The normal phase transmission signal string and the reversed phase transmission signal string are in a phase inversion relationship. The same transmission weighting is applied to the two transmission signal strings. A basic wave component is suppressed and a harmonic wave component is extracted by adding the two beam data obtained by two times of transmission/reception. The harmonic wave component may be extracted by a method other than the PI method.

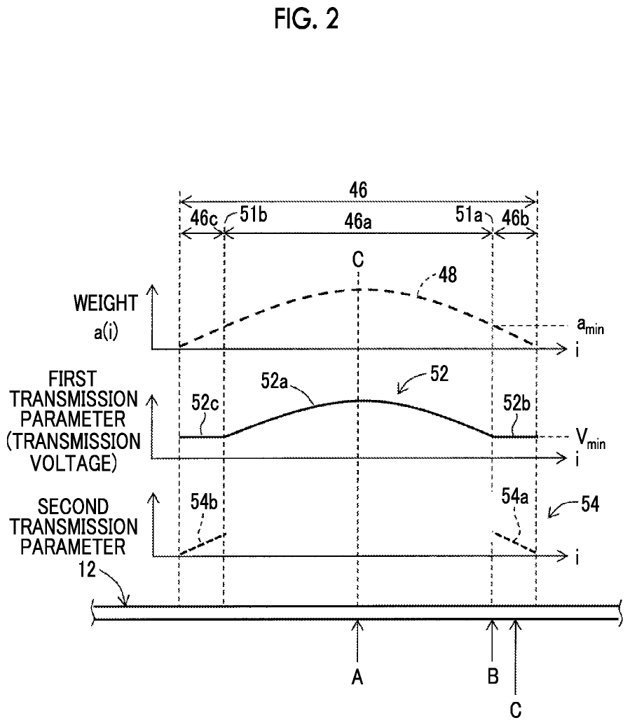

FIG. 2 shows the transmission weighting method according to the embodiment. C indicates an aperture center. A horizontal axis indicates a position i of the oscillator. The transmission aperture 46 is set for the oscillator array 12.

The transmission circuit has a voltage range in which the voltage can be appropriately generated, and a voltage lower than a lower limit Vₘᵢₙ of the voltage range cannot be generated or the generation of such a low voltage should be avoided. In a case where the voltage varying method is selected as a main transmission weighting method (first transmission weighting method), the voltage range should be taken into consideration.

Therefore, in the embodiment, the transmission aperture is divided into an intermediate portion 46a, a first end portion 46b, and a second end portion 46c. The main transmission weighting method of varying the transmission voltage is applied to the intermediate portion 46a, and a sub-transmission weighting method of varying the transmission parameter (second transmission parameter) other than the transmission voltage (first transmission parameter) is applied to the first end portion 46b and the second end portion 46c under the condition that the transmission voltage is made constant. A boundary between the intermediate portion 46a and the first end portion 46b is a first boundary 51a, and a boundary between the intermediate portion 46a and the second end portion 46c is a second boundary 51b.

FIG. 2 shows a transmission voltage distribution (first transmission parameter distribution) 52 as the weight distribution in accordance with the main transmission weighting method, and a second transmission parameter distribution 54 as the weight distribution in accordance with the sub-transmission weighting method. The transmission voltage distribution 52 is applied to the entire transmission aperture 46. The second transmission parameter distribution 54 is limitedly applied to the first end portion 46b and the second end portion 46c. The second transmission parameter is the parameter other than the transmission voltage, is a parameter that affects an amplitude of the ultrasound pulse transmitted from the oscillator array, and is, for example, the duty (pulse width) or the wave number, which will be described later. The second transmission parameter can be referred to as a complementary weight.

Specifically, the transmission voltage distribution 52 consists of a portion 52a applied to the intermediate portion 46a, a portion 52b applied to the first end portion 46b, and a portion 52c applied to the second end portion 46c. The portion 52a is a portion following an ideal weight distribution 48. The portion 52b and the portion 52c are portions each having a certain voltage corresponding to the lower limit Vₘᵢₙ. Specifically, the transmission voltage distribution 52 is represented as follows.

$$V ( i ) = \{ \begin{matrix}V ( i ) , & if a ( i ) > {a}_{min} \\ {V}_{min} , & else\end{matrix} \qquad ( 1 )$$

By adopting the transmission voltage distribution 52, it is possible to avoid the setting of the voltage lower than the lower limit Vₘᵢₙ for the first end portion 46b and the second end portion 46c.

The second transmission parameter distribution 54 consists of a portion 54a applied to the first end portion 46b, and a portion 54b applied to the second end portion 46c. Both the portion 54a and the portion 54b can be said to be portions that follow the ideal weight distribution 48 or portions that complementarily realize the ideal weight distribution.

According to the embodiment, by using the main transmission weighting method and the sub-transmission weighting method in combination, in other words, by using the combination of the transmission voltage distribution 52 and the second transmission parameter distribution 54, it is possible to bring the sound pressure distribution of the ultrasound waves radiated from the transmission aperture close to the ideal weight distribution 48. The transmission weighting control is performed such that an actual sound pressure distribution is smoothly connected at the boundaries 51a and 51b.

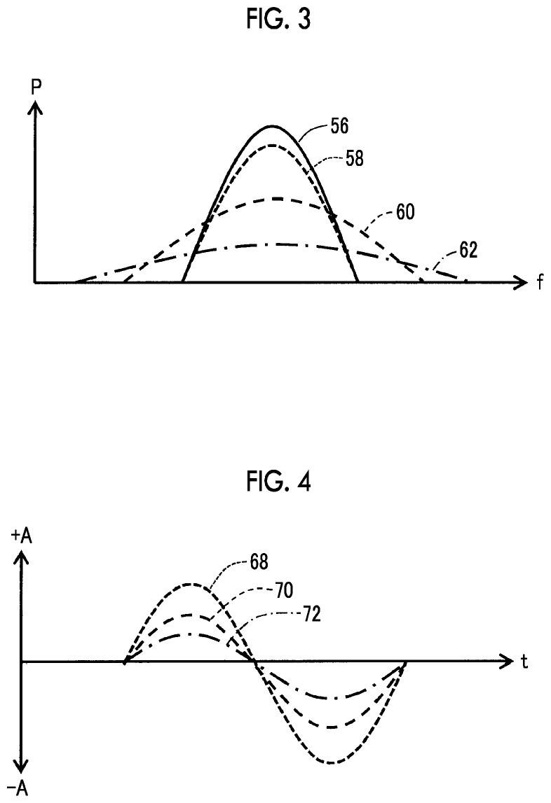

An example of realizing the weight for varying the second transmission parameter will be described with reference to FIGS. 3 and 4. In this example, the second transmission parameter is, for example, the wave number. FIG. 3 is a diagram showing a plurality of frequency characteristics (signal band) 58, 60, and 62 included in the plurality of transmission signals. A horizontal axis is a frequency axis, and a vertical axis is a power axis. Reference numeral 56 denotes a frequency characteristic (pass band) of the oscillator array. In a case where the plurality of transmission signals are supplied to the oscillator array, the pass band 56 acts on the plurality of signal bands 58, 60, and 62.

As a result, three sound pressure signals shown in FIG. 4 are generated. A horizontal axis is a time axis, and a vertical axis is an amplitude axis (sound pressure axis). A sound pressure signal 68 corresponds to the signal band 58 shown in FIG. 3, a sound pressure signal 70 corresponds to the signal band 60 shown in FIG. 3, and a sound pressure signal 72 corresponds to the signal band 62 shown in FIG. 3. As shown in FIGS. 3 and 4, the amplitude of the sound pressure waveform can be operated by varying the signal band (specifically, a signal band width). That is, the transmission weighting can be performed by varying the signal bandwidth.

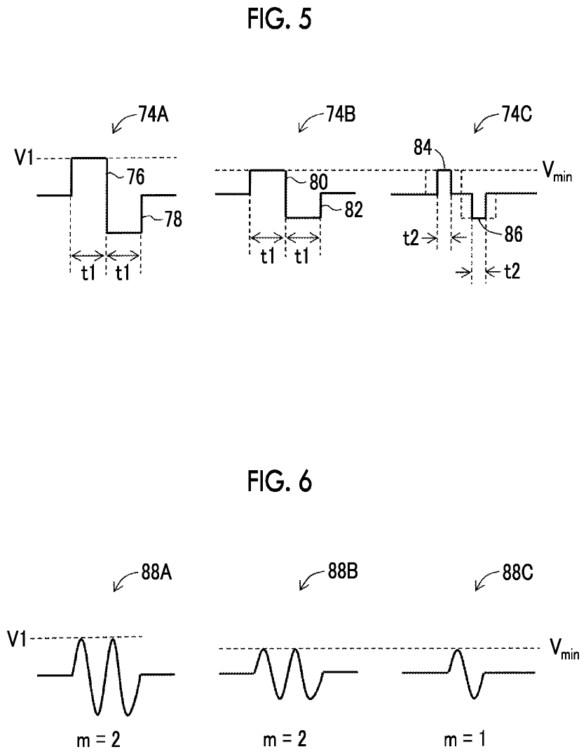

FIG. 5 shows a first example of three transmission signals supplied to three positions A, B, and C in the transmission aperture 46 shown in FIG. 2.

In the first example shown in FIG. 5, a pulse width varying method is adopted as the sub-transmission weighting method. A transmission signal 74A supplied to the position A includes a positive pulse 76 and a negative pulse 78, a height of the positive pulse 76 is +V1, and a height of the negative pulse 78 is −V1. +V1 (and −V1) is the transmission voltage (maximum transmission voltage) designated by the user or automatically set. The widths of the positive pulse 76 and the negative pulse 78 are all t1.

A transmission signal 74B supplied to the position B includes a positive pulse 80 and a negative pulse 82, a height of the positive pulse 80 is +Vₘᵢₙ (<+V1), and a height of the negative pulse 82 is −Vₘᵢₙ. The widths of the positive pulse 80 and the negative pulse 82 are all t1.

A transmission signal 74C supplied to the position C includes a positive pulse 84 and a negative pulse 86, a height of the positive pulse 84 is Vₘᵢₙ, and a height of the negative pulse 86 is −Vₘᵢₙ. The widths of the positive pulse 84 and the negative pulse 86 are all t2 (<t1).

In the end portion, the pulse width is gradually decreased from the boundary to the end portion while the lower limit Vₘᵢₙ is maintained, that is, the duty is gradually decreased. As a result, it is possible to realize appropriate weighting even in the end portion.

FIG. 6 shows a second example of the three transmission signals supplied to the three positions A, B, and C in the transmission aperture 46 shown in FIG. 2.

In a second example shown in FIG. 6, a wave number varying method is adopted as the sub-transmission weighting method. A transmission signal 88A supplied to the position A consists of two waves. That is, the wave number m is 2. An amplitude on a positive side is +V1, and an amplitude on a negative side is −V1. A transmission signal 88B supplied to the position B also consists of two waves. That is, the wave number m is 2. An amplitude on a positive side is +Vₘᵢₙ (<V1), and an amplitude on a negative side is −Vₘᵢₙ (<V1). A transmission signal 88C supplied to the position C consists of one wave. That is, the wave number m is 1. An amplitude on a positive side is +Vₘᵢₙ, and an amplitude on a negative side is −Vₘᵢₙ. A numerical value having a decimal number may be set as the wave number m.

In the end portion, the wave number is gradually decreased from the boundary to the end portion while the lower limit Vₘᵢₙ is maintained, in other words, the band is gradually widened. As a result, appropriate weighting is realized even in the end portion.

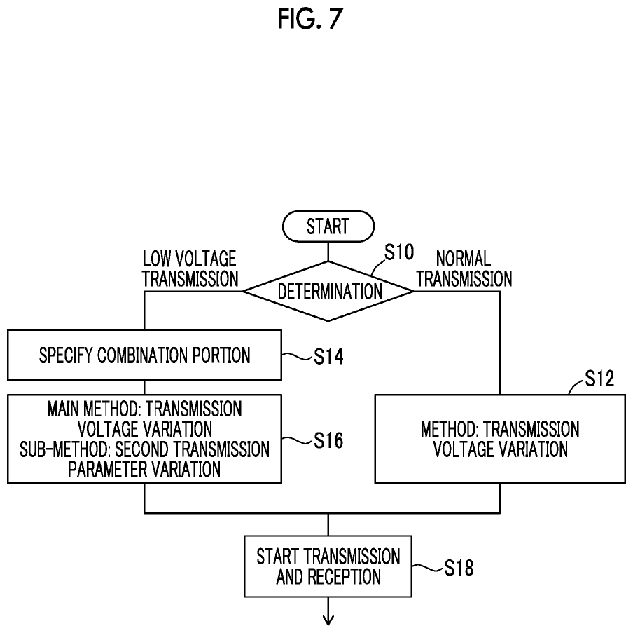

FIG. 7 shows an operation example of the ultrasound diagnostic apparatus shown in FIG. 1. In S10, the representative transmission voltage in the transmission signal string is compared with the threshold value. The representative transmission voltage is, for example, the transmission voltage designated by the user or a preset transmission voltage. In a case where the representative transmission voltage is higher than the threshold value, that is, in a case of the normal transmission, the transmission voltage varying method is selected as the transmission weighting method applied to the entire transmission aperture in S12. Thereafter, in S18, the transmission and reception of the ultrasound waves are started.

On the other hand, in S10, in a case where it is determined that the representative transmission voltage is lower than the threshold value, that is, in a case where the low voltage transmission is determined, the intermediate portion, the first end portion, and the second end portion in the transmission aperture are specified in accordance with the ideal weight distribution and the minimum transmission voltage (lower limit) in S14. In S16, the transmission voltage (first transmission parameter) varying method is selected as the main transmission weighting method applied to the intermediate portion, and the second transmission parameter varying method is selected as the sub-transmission weighting method applied to the first end portion and the second end portion. Thereafter, in S18, the transmission and reception of the ultrasound waves are started.

In S10, the normal transmission or the low voltage transmission may be determined in accordance with the transmission/reception mode (operation mode). For example, the low voltage transmission may be determined in the CHI mode, and normal transmission may be determined in the other modes.

A method other than the voltage varying method may be adopted as the main transmission weighting method. A method of varying the transmission parameter other than the duty and the wave number may be adopted as the sub-transmission weighting method. The transmission weighting method according to the embodiment can be applied in a case where a portion for performing the low voltage transmission and the other portion are set in the transmission aperture. In this case, a portion other than the end portion may be the portion for performing the low voltage transmission.

The present application claims priority from Japanese Patent Application No. 2023-075809 filed on May 1, 2023, the content of which is hereby incorporated by reference into this application.

### EXPLANATION OF REFERENCES

10: ultrasound probe
12: oscillator array
14: transmission circuit
16: transmitter
32: reception circuit
42: main controller
44: transmission/reception controller
46: transmission aperture
46a: intermediate portion
46b: first end portion
46c: second end portion
48: ideal weight distribution
52: voltage distribution
54: waveform parameter distribution

What is claimed is:

1. An ultrasound diagnostic apparatus comprising:
an oscillator array;
a transmission circuit that supplies a transmission signal string to a transmission aperture set in the oscillator array; and
a controller that controls generation of each transmission signal constituting the transmission signal string,
wherein the transmission aperture has a first portion and a second portion, and
the controller applies a first transmission weighting method to a plurality of transmission signals supplied to the first portion, and applies a second transmission weighting method different from the first transmission weighting method to a plurality of transmission signals supplied to the second portion.

2. The ultrasound diagnostic apparatus according to claim 1,
wherein the first portion is an intermediate portion in the transmission aperture, and
the second portion is a first end portion and a second end portion provided on both sides of the intermediate portion in the transmission aperture.

3. The ultrasound diagnostic apparatus according to claim 2,
wherein the first transmission weighting method is a transmission weighting method of varying a transmission voltage, and
the second transmission weighting method is a transmission weighting method of varying a transmission parameter other than the transmission voltage.

4. The ultrasound diagnostic apparatus according to claim 3,
wherein, in the first transmission weighting method, the transmission voltage is varied over a range from a first transmission voltage to a second transmission voltage lower than the first transmission voltage,
voltages of the plurality of transmission signals supplied to the first end portion and the second end portion are all the second transmission voltages, and
waveforms of the plurality of transmission signals supplied to the first end portion and the second end portion are set in accordance with the second transmission weighting method.

5. The ultrasound diagnostic apparatus according to claim 4,
wherein the transmission aperture has a first boundary between the intermediate portion and the first end portion, and a second boundary between the intermediate portion and the second end portion, and
the controller controls variation of the transmission voltage and variation of the transmission waveform such that a convex sound pressure distribution corresponding to the transmission aperture is substantially continuous at the first boundary and the second boundary.

6. The ultrasound diagnostic apparatus according to claim 3,
wherein the transmission parameter other than the transmission voltage is a pulse width, and
the controller varies the pulse width of each transmission signal in a case of application of the second transmission weighting method.

7. The ultrasound diagnostic apparatus according to claim 3,
wherein the transmission parameter other than the transmission voltage is a wave number, and
the controller varies the wave number of each transmission signal in a case of application of the second transmission weighting method.

8. The ultrasound diagnostic apparatus according to claim 2,
wherein the controller
applies the first transmission weighting method to a plurality of transmission signals supplied to an entire transmission aperture in a case where a representative transmission voltage of the transmission signal string is higher than a threshold value, and
applies the first transmission weighting method to the plurality of transmission signals supplied to the intermediate portion and applies the second transmission weighting method to the plurality of transmission signals supplied to the first end portion and the second end portion in a case where the representative transmission voltage is lower than the threshold value.

9. A transmission beam forming method comprising:
a generation step of generating a transmission signal string for forming a transmission beam; and
a supply step of supplying the transmission signal string to a transmission aperture set in an oscillator array,
wherein the transmission aperture has a first portion and a second portion, and
in the generation step, a first transmission weighting method is applied to a plurality of transmission signals supplied to the first portion, and a second transmission weighting method different from the first transmission weighting method is applied to a plurality of transmission signals supplied to the second portion.
